(19) United States
(12) Patent Application Publication
(10) Pub. No.: US 2026/0188504 A1
(43) Pub. Date: Jul. 2, 2026

(54) CONTACT CENTER INTELLIGENT DATA RETRIEVAL SYSTEM AND METHOD

(71) Applicant: Cigna Intellectual Property, Inc., Wilmington, DE (US)

(72) Inventor: Oliver L. Ilagan, Nashville, TN (US)

(21) Appl. No.: 19/007,111

(22) Filed: Dec. 31, 2024

(51) Int. Cl. G16H 50/30; G06N 3/02; G16H 20/00

(52) U.S. Cl. CPC G16H 50/30; G06N 3/02; G16H 20/00

(57) ABSTRACT

A computer-implemented method for enhancing call center operations (e.g., for a pharmacy benefit manager) includes recording inputs from members or call center agents, providing these inputs to a large language model (LLM), accessing various healthcare-related databases using the LLM, identifying responsive information from these databases, and communicating this information back to the call center agent device to prevent the agent from manually searching through different databases.

### BACKGROUND

Call centers are used in a variety of industries, including management of benefit plans (e.g., pharmacy benefit plans), sales, customer support, etc. Human agents at these call centers can be required to handle a wide variety of inquiries from members (e.g., of benefit plans), providers (e.g., of services, such as healthcare providers), customers, or the like, that call into the centers. These agents can spend a significant amount of time scavenging for data across many sources to serve the callers. Agents may be required to manually gather data from various tools and applications to understand the context of the data, fetch the data, aggregate the data, and then communicate the data back to the caller. This manual process places a significant burden on the agents, requiring extensive training and leading to inefficiencies in handling customer inquiries.

This can distract the agents from focusing on the callers, which can imply a lack of empathy to the callers and result in unsatisfactory customer or member experiences. For example, the agents may be required to access different databases via different tools or web interfaces, which in turn can require the agents to repeatedly enter the same or different identifying information about the callers in different tools to interfaces, to obtain information from one database only to acquire additional information needed for the inquiry from another database, and so on. This can involve a significant amount of mental effort that can distract the agent from the caller.

Additionally, training of new agents for call centers can be a significant undertaking both in terms of time and effort due to these complexities. For example, the majority of onboarding for new agents can be spent training the agents simply on how to navigate between and within the many data sources or databases that the agents much review (e.g., around twelve weeks plus two additional weeks with database access provisioning or management).

A need exists for improving the workflow of call center agents to respond to caller inquiries faster, improve the caller experience, and reduce the burden on training new agents.

Additionally, customers or members (e.g., of benefit plans) may have many encounters over time. For example, a pharmacy benefit plan member may have many healthcare provider visits, pharmacy benefit claims, medical procedures, or the like. Each of these encounters generates data that may be needed for future encounters. Some medical procedures or prescriptions may require preauthorization, which can involve ensuring that certain data or events are collected before benefits for the procedures or prescriptions are authorized. As another example, some benefit claims may be denied, and appealing the denial of the benefit claims can be data-intensive and complicated process for members. Because the benefit plan members are typically not experts at the data collection practices and some requirements for benefits, members may be unable to navigate the processes of the benefit plan manager to obtain the benefits or preauthorizations.

A need exists for pro-actively assisting members of benefit plans in handling the large amounts of information needed to acquire benefits under the plans.

### BRIEF SUMMARY

In one example, a computer-implemented method for enhancing call center operations (e.g., for a pharmacy benefit manager) includes recording inputs from members or call center agents, providing these inputs to a large language model (LLM), accessing various healthcare-related databases using the LLM, identifying responsive information from these databases, and communicating this information back to the call center agent device to prevent the agent from manually searching through different databases. The LLM can further present or recommend predefined set of actions/automations based on the context of the interaction determined.

In another example, an application-specific integrated circuit (ASIC) for an artificial neural network (ANN) includes a large language model (LLM). The ASIC comprises neurons organized in an array, each with a register, a processing element, and at least one input. Synaptic circuits connect the neurons, each storing a synaptic weight. The processing elements are configured to record inputs from call center agents or members, access healthcare-related databases, identify responsive information, and communicate this information back to the call center agent device to streamline the process and reduce the need for manual searches.

In another example, an ASIC for an ANN that functions as an automated healthcare data concierge. The ASIC includes neurons organized in an array, each with a register, a processing element, and at least one input, connected by synaptic circuits storing synaptic weights. The processing elements are configured to record data from member enrollments and healthcare provider visits, generate or update a ledger of this data, identify claims for benefits, determine the best support channel for resolving claims, access pre-aggregated healthcare data, and automatically resolve claims using this data. In an example, the processing elements can further engage workflows to gather additional information, which can be determined as needed by the LLM, to resolve the issue.

### BRIEF DESCRIPTION OF THE SEVERAL VIEWS OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some embodiments are illustrated by way of example, and not limitation, in the figures of the accompanying drawings in which:

FIG. 1 illustrates one example of a call center operations system;

FIG. 2 illustrates one example of a machine learning (ML)/artificial intelligence (AI) system;

FIG. 3 illustrates a flowchart of one example of a method for operating a call center;

FIG. 4 illustrates one example of an automated healthcare data concierge system;

FIG. 5 illustrates one example of operation of the automated healthcare data concierge system shown in FIG. 4; and

FIG. 6 illustrates a flowchart of one example of a method for operating an automated healthcare data concierge system.

### DETAILED DESCRIPTION

The description that follows includes systems, methods, techniques, instruction sequences, and computing machine program products that embody illustrative embodiments of the disclosure. In the following description, for the purposes of explanation, numerous specific details are set forth in order to provide an understanding of various embodiments of the inventive subject matter. It will be evident, however, to those skilled in the art, that embodiments of the inventive subject matter may be practiced without these specific details. In general, well-known instruction instances, protocols, structures, and techniques are not necessarily shown in detail.

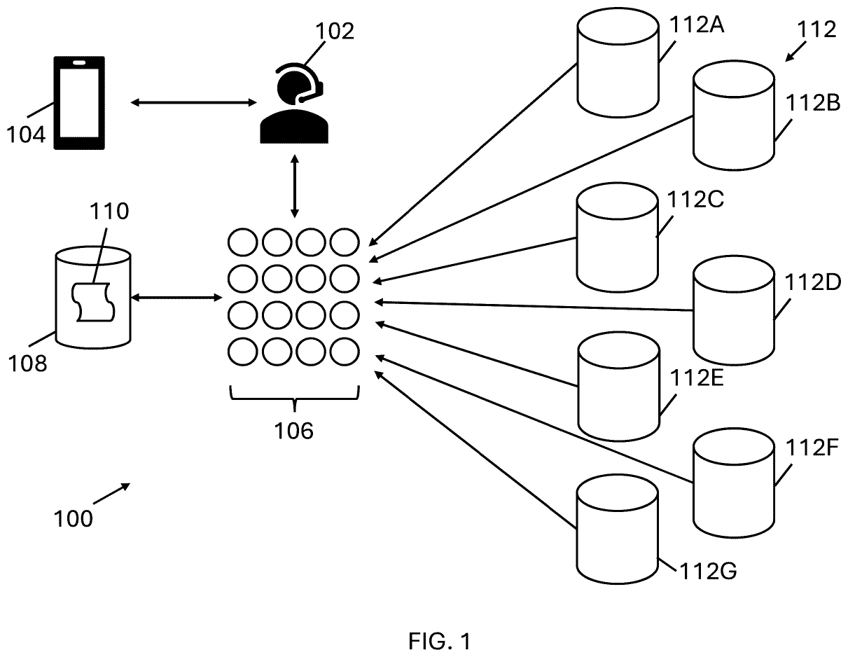

FIG. 1 illustrates one example of a call center operations system 100. The operations system 100 includes one or more (or many) call center agent devices 102 that receive inquiries from customer or member devices 104. Each of the devices 102, 104 can represent hardware circuitry that includes and/or is connected with one or more processors for performing the operations described in connection with each of the devices 102, 104. The processor(s) can include one or more integrated circuits, ASICs, microprocessors, field programmable gate arrays, etc. The devices 102, 104 include communication circuitry such as antennas, modems, converters, etc. that can communicate with each other via or over one or more computerized communication networks (e.g., the Internet, one or more intranets, wide area networks, local area networks, etc.).

The operations center 100 includes an artificial intelligence (AI) system, a machine learning (ML) system, and/or an ANN 106 that performs operations described herein. The ANN 106 can assist a call center agent via the agent device 102. For example, callers may submit inquiries to the call center using the member devices 104. These inquiries can be phone calls, text messages or other messaging services, online chats, or the like, between the devices 102, 104. The inquiries can include a variety of questions seeking assistance, such as seeking information regarding one or more benefits provided by a pharmacy benefit plan. For example, members may contact the agent device 102 asking whether a prescription for medication or a treatment is covered by benefits under a benefit plan, why a claim for benefits under the plan was denied, whether preauthorization for a benefit under the plan is required, what remaining information is required for the preauthorization, where or how the member can get the information or remaining information needed for preauthorization, whether the member can appeal denial of a benefit claim, how to appeal the denial of a benefit claim, what information is required for the appeal, where or how the member can get the information or remaining information needed for the appeal, and so on.

An artificial intelligence (AI) system, machine learning (ML) system, or ANN 106 of the operations system 100 can receive inputs provided to the agent device 102. These inputs can include the ANN 106 recording an audio call between the devices 102, 104 and transcribing the call into text or alphanumeric text. The inputs can include the typed messages sent from the member device 104 or between the devices 102, 104. The inputs can include keystrokes, electronic mouse inputs, electronic stylus inputs, detected touches on a touchscreen, or the like, on the agent device 102. The ANN 106 can record these and/or other inputs in a tangible and non-transitory computer readable storage medium 108, or computer memory 108. This memory 108 can represent one or more computer hard drives, servers, databases, optical discs, or the like.

The ANN 106 can provide the recorded inputs to an LLM 110 of the operations system 100. The LLM 110 may be a model stored in the memory 108 or in another tangible and non-transitory computer readable storage medium. The LLM 110 can be trained to identify the context of the inquiry from the member device 104, as well as information that may be responsive to the inquiry. The LLM 110 can receive the recorded inputs of the transcribed call, messages, keystrokes by the agent on the agent device 102, etc. and, based on these inputs, determine what information will assist the agent in rectifying or otherwise responding to the inquiry. For example, the LLM 110 can examine the recorded inputs and decide that the inquiry relates to whether a certain medication or medical procedure is covered by a benefit provided pursuant to a benefit plan. These inputs may include terms such as “covered,” “coverage,” “am I,” names of medications, names of providers, or the like. As another example, the LLM 110 can examine the recorded inputs and decide that the inquiry relates to why a benefit claim was denied. These inputs may include terms such as “why,” “denied,” names of medications, dates of denials, or the like. As another example, the LLM 110 can examine the recorded inputs and decide that the inquiry relates to what information is needed for preauthorization of benefits for a medication or medical therapy/treatment. These inputs can include terms such as “preauthorization,” “preauthorized,” etc.

Once the purpose of the inquiry is identified by the LLM 110, the LLM 110 can output the inquiry purpose to the ANN 106. The ANN 106 can then automatically access one or more, or several, different databases 112 (e.g., databases 112A-G) to obtain information helpful to responding to the inquiry. While seven databases 112 are shown, fewer or more databases 112 may be accessible to the ANN 106. These databases 112 can each represent a tangible and non-transitory computer readable storage medium, or can represent different sections or partitions within the same storage medium. The databases 112 may be maintained by the operations system 100 or by a third party, such as a healthcare provider, hospital system, or the like.

The database 112A can represent a benefit claims database that stores medical claims data. The claims data can include information regarding pharmacy claims adjudicated by the pharmacy benefit manager under a drug benefit program provided by the pharmacy benefit manager for one, or more than one, plan sponsors. The claims data can identify the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copay/coinsurance amount, rebate information, and/or member eligibility, and the like. Additional information may be included.

Other types of claims beyond prescription drug claims may be stored in the claims data. For example, medical claims, dental claims, wellness claims, or other type of health care-related claims for members may be stored as a portion of the claims data. In some embodiments, the claims data includes claims that identify the members with whom the claims are associated. The claims data can include claims that have been de-identified (e.g., associated with a unique identifier but not with a particular, identifiable member), aggregated, or otherwise processed.

The database 112B can represent a dental claims database storing dental benefit claims data. This claims data may be similar or identical to the claims data stored in the database 112A except related to dental procedures and claims.

The database 112C can represent a provider database storing healthcare provider data. This data can indicate the names, locations, practice areas, etc. of different healthcare providers, whether the providers are within a provider network of a benefit plan for members or outside of the network, and the like. Optionally, this data can indicate prior claims, procedures, preauthorizations, failed preauthorizations, or the like, involving different healthcare providers.

The database 112D can represent a member database storing member data. The member data can include information regarding the members associated with the pharmacy benefit manager. The information stored as member data may include personal information, personal health information, protected health information, and the like. Examples of the member data include demographic information such as names, addresses, telephone numbers, e-mail addresses, prescription drug histories, etc., and the like. The member data may include a plan sponsor identifier that identifies the plan sponsor associated with the member and/or a member identifier that identifies the member to the plan sponsor. The member data may include a member identifier that identifies the plan sponsor associated with the patient and/or a patient identifier that identifies the patient to the plan sponsor. The member data may also include, by way of example, dispensation preferences such as type of label, type of cap, message preferences, language preferences, or the like.

The database 112E can represent a pharmacy database that stores pharmacy claims data. This claims data may be similar or identical to the claims data stored in the database 112A except related to claims submitted to pharmacies.

The database 112F can represent a plan database storing benefit plan eligibility data. The eligibility data can include claim adjudication rules or criteria used to decide whether a medication or treatment claim is covered by a benefit plan, whether certain providers are within a benefit network provided by the benefit plan, what details are needed for preauthorization of a benefit claim to be approved, what details are needed to appeal a denial of a claim, etc.

The database 112G can represent a documentation database that stores documents or logs. These documents can detail the eligibility data stored in the database 112F, can store logs from prior interactions between the agent device 102 and member devices 104 (e.g., a history of calls or messages between the devices 102, 104), prior actions taken in response to an inquiry from a member to the agent device 104, or the like.

The ANN 106 can access one or more of the databases 112 based on the recorded inputs and the context of the inquiry as determined by the ANN 106. The ANN 106 can obtain one or more sets of responsive information from the healthcare-related databases 112 based on the inputs. For example, in response to the ANN 106 deciding that the inquiry is asking about one or more benefits provided by a pharmacy benefit plan, the ANN 106 can access the database 112F to obtain eligibility data for the benefit(s) and/or the database 112G to obtain documents summarizing or describing the benefits provided under a plan.

As another example, in response to the ANN 106 deciding that the inquiry is asking whether a prescription for medication or a treatment is covered by benefits under a benefit plan, the ANN 106 can access the database 112F to obtain eligibility data for the prescription and/or the database(s) 112A, 112B, 112E to see whether prior benefits were provided to the member or other member(s) pursuant to the plan.

As another example, in response to the ANN 106 deciding that the inquiry seeks information on why a claim for benefits under the plan was denied, the ANN 106 can access the database(s) 112A, 112B, 112E to obtain details on why the specific claim was denied, as well as the database 112F to determine the eligibility of the member for benefits to the claim and/or the database 112G to review prior logs regarding the adjudication of the claim that was denied.

In response to the ANN 106 deciding that the inquiry seeks guidance on whether preauthorization for a benefit under the plan is required, the ANN 106 can access the database(s) 112A, 112B, 112E to examine prior similar or identical claims and decide whether preauthorization was required in those prior claims (as well as what information was needed to obtain the preauthorization). The ANN 106 can access the plan eligibility data to obtain information on whether preauthorization is needed and what information is required for obtaining the preauthorization from the database 112F, as well as the database 112G to examine prior histories of the member to see whether preauthorization was previously required for the benefit claim.

The ANN 106 can access the database 112F to examine appeal procedures responsive to the inquiry seeking information on appealing a denial of a benefit claim. The ANN 106 also can access the database 112G to obtain any forms required to be filled out and submitted with the appeal, as well as one or more other databases 112 to gather the information needed to complete the forms and submit the appeal.

Once the information is gathered by the ANN 106, the ANN 106 can aggregate the data obtained from the databases 112, generate one or more electronic signals, and send the signal(s) to the agent device 102. These signals are sent to communicate or convey the set(s) of responsive information obtained by the ANN 106 to the agent device 102. This can provide a single source or interface for the agent device 102 to obtain the information that responds to the caller inquiry from the member device 104. In contrast, the agent using the agent device 102 may be required to successively access, in a serial manner, each database 112 containing (or that the agent believes may contain) information responsive to the inquiry. Accessing each database 112 can require separate login and password entries, clicking through multiple interfaces, and the like, to obtain the responsive information from that database 112 alone. Additionally, the agent may mistakenly believe that a database 112 may have responsive information and spend additional time accessing and searching through that database 112 for the information that is not in the database 112. All of this can consume a significant amount of time and distract the agent from listening or otherwise interacting with the member.

In one example, the ANN 106 can automatically complete one or more appeal forms for appealing a denial of a claim for benefit under the pharmacy benefit plan based on the responsive information that is obtained. For example, responsive to determining that the inquiry from the member device 104 is asking about a claim denial and/or an appeal of a claim denial, the ANN 106 can obtain the necessary forms for appealing the claim denial and the information needed to appeal the claim denial from the databases 112. The ANN 106 can then pre-populate the forms with the needed information before sending and presenting this form and information to the agent device 102. This can allow the agent to discuss the appeal with the member with the agent having more knowledge about the appeal and can quickly identify any information that is needed but missing from the forms. In one example, the ANN 106 can automatically submit the one or more appeal forms to the pharmacy benefit manager using appeal the denial of the claim on behalf of the member. This can result in an improvement in the functioning of the operations system 100 in that a denial for a medical procedure or medication (resulting in a patient being denied needed treatment or medication) can be reversed more quickly and more efficiently so that the patient can receive the needed treatment or medication sooner.

The ANN 106 can record the communications between the agent device 102 and the member device 104, as described above. Additionally, the ANN 106 can create a summary of these recorded communications, as well as the responsive information obtained by the ANN 106. The ANN 106 can send a signal to the agent device 102 that directs the agent device 102 (e.g., an electronic display device of the agent device 102, such as a computer monitor, touchscreen, or the like) to present the summary to the call center agent via the agent device 102. The call center agent can then review the summary and either approve or reject the summary. For example, if the summary accurately reflects the interaction with the member, then the agent can provide input into the agent device 102 indicating validation or approval of the summary (referred to as a validation action). This validation action can be communicated to the ANN 106, which can then populate a call log with the summary and store the call log in the database 112G for further use in future calls.

While one example of the operations system 100 can be used in connection with member devices 104 contacting the agent device(s) 102 for real-world or actual inquiries, another example of the operations system 100 can be used in connection with training an agent to efficiently and effectively use the operations system 100 for handling real-world or actual inquiries. For example, recordings of prior member calls, hypothetical inquiries, or the like, can be presented to the agent via the agent device 102, and the ANN 106 can operate as described above to provide the responsive information to the agent device 102 during training of the agent (and/or refinement of the ANN 106).

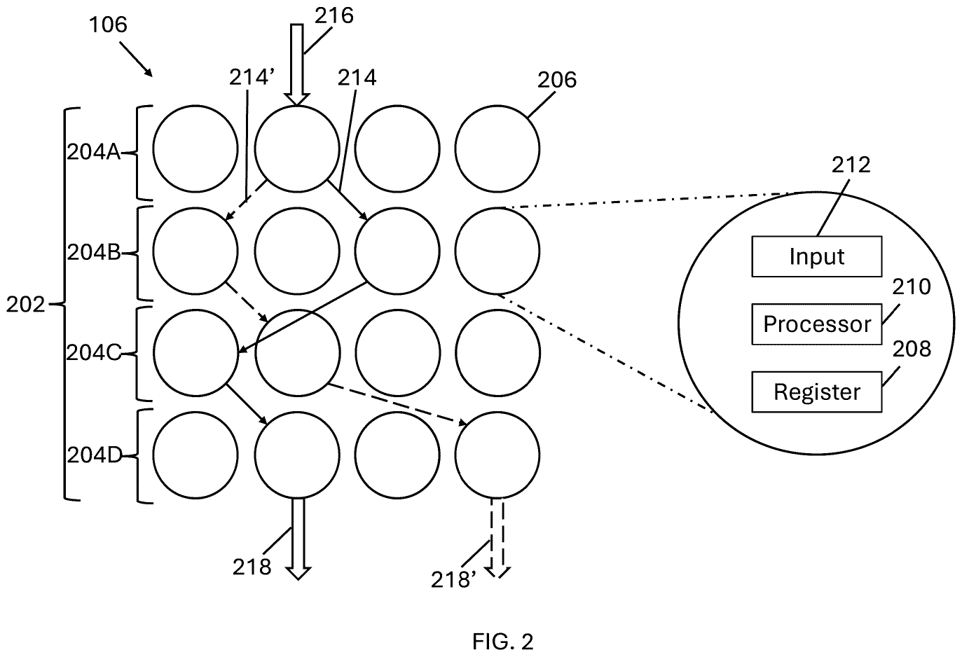

FIG. 2 illustrates one example of the ANN 106 shown in FIG. 1. The ANN 106 can be embodied in one or more ASICs for the ANN 106. The ASIC(s) can includes a series 202 of layers 204A-D, each comprising one or more artificial neurons 206 arranged in one or more neuron arrays or arrangements. While four neurons 206 are shown in each layer 204A-D and four layers 204A-D are shown, alternatively, a different number of neurons 206 may be in one or more of the layers 204A-D and/or there may be a different number of layers 204A-D.

The ANN 106 may include the neurons 206 arranged in an input layer 204A, an output layer 204D, and two or more fully connected hidden or intermediate layers 204B, 204C between the input and output layers 204A, 204D. Each neuron 206 can include or represent a register 208, a microprocessor or processing element 210, and at least one input 212. The neurons 206 can generate outputs based on one or more activation functions. The neurons 206 can receive input from another neuron 206 (e.g., the output from one neuron 206 can be the input for another neuron 206). This input also can include a set of weights. The neurons 206 can be connected with each other via synaptic circuits 214, 214′. The synaptic circuits 214, 214′ can include or represent memories for storing synaptic weights.

One or more neurons 206 (e.g., nodes) in the input layer 204A of the ANN 106 can receive an input 216 into the ANN 106. The input 216 can include, for example, the recorded inputs of a call or interaction between the devices 102, 104 shown in FIG. 1. The neurons 206 can receive this input data 216 via the input(s) 212 of the neurons 206 in the input layer 204A. The neurons 206 receive the input data 216, apply one or more mathematical equations or relationships stored in the registers 208 (and that include the weights) to generate an output. The processors 210 of the neurons 206 apply the equations/relationships and can pass the output to another neuron 206 in the same layer 204A or in a different layer 204B, 204C. The output from one neuron 206 is passed along a synaptic circuit 214 to another neuron 206 and is used as input to this other neuron 206. This process continues until one or more neurons 206 in the output layer 204D generate an output 218 from the ANN 106.

The synaptic circuits 214, 214′, weights stored in the synaptic circuits 214, 214′, and/or the mathematical relationships between the neurons 206 can define the LLM 110. In operation, the ANN 106 can operate by converting the recorded inputs into a machine-readable summary. This conversion can be performed by modifying the recorded inputs into series of tokens via a tokenization process. The different tokens can represent words in the recorded inputs, portions of words such as morphemes, phrases formed of multiple words, and the like. The synaptic circuits 214, mathematical equations, and weights defining the model 110 may dictate what tokens are generated based on the recorded inputs. Changing the model 110 by changing which neurons 206 are connected by which synaptic circuits 214, which weights are applied to different words or inputs into the recorded inputs to select which words or phrases are included in the tokens, and the like, can change the sequence of tokens output based on the recorded inputs. For example, the LLM 110 can output a first sequence of tokens but output a different, second sequence responsive to the LLM 110 being modified (e.g., refined).

The ANN 106 can then map the token sequence into vector space. During training of the LLM 110, the ANN 106 using the LLM 110 can encounter words and examine the different contexts of the words as well as relationships with other words. ANN 106 can assign a unique vector to each word in the training data based on the LLM 110 during this training. These single word assignments can be used by the ANN 106 to create assignments of text documents to unique vectors in the vector space. After training, the ANN 106 can use the LLM 110 to assign different vectors to different sequences of tokens. Each vector can define a location within the vector space, and words and/or phrases with similar or identical meanings may have locations that are closer in the vector space than words and/or phrases with more dissimilar or different meanings. Additionally, the distance and direction between vectors within the vector space can represent relationships between the words and/or phrases indicated by the token sequences. For example, the distance and direction between vectors may reflect semantic relationships between the words and/or phrases associated with the token sequences.

This vector space may then be used by the ANN 106 to identify context of inquiries from member devices 104, as well as to determine what responsive information is needed from the databases 112. For example, certain vectors are associated with different sets of responsive information. If the tokens from a recorded input is closer to one vector than others in the vector space defined by the LLM 110, then the ANN 106 can obtain the responsive information from the databases 112 that is associated with that vector. The LLM 110 can be refined based on feedback. For example, if the returned responsive information is not responsive to the inquiry, feedback identifying the additional or different responsive information that is needed can be provided to the ANN 106. The ANN 106 can then modify the circuits, weights, or the like, of the LLM 110 so that a different vector is associated with the same recorded input as before. This can allow the LLM 110 to continue to improve over time.

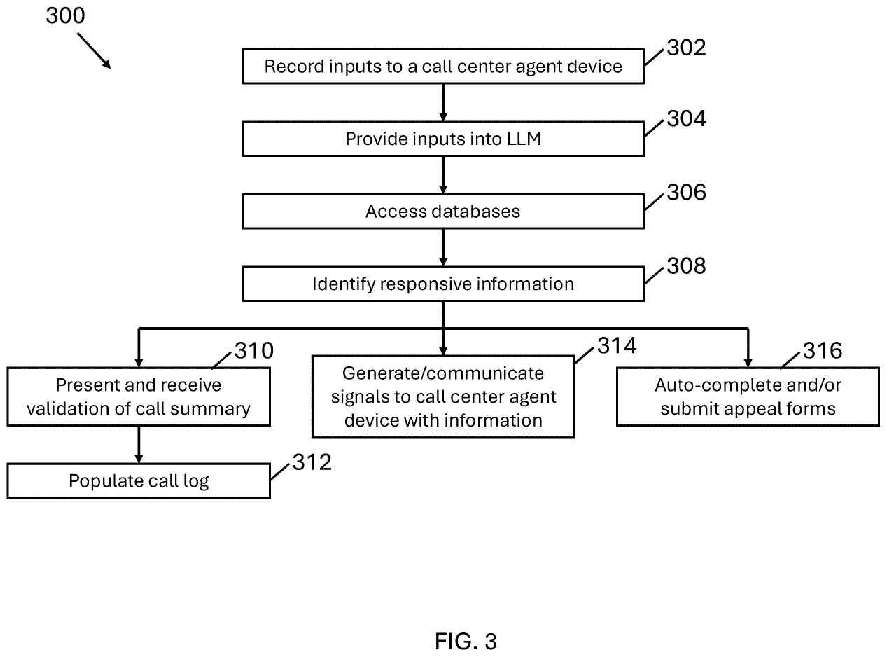

FIG. 3 illustrates a flowchart of one example of a method 300 for data retrieval in a call center operations system. The method 300 can represent operations performed by the operations system 100 shown in FIG. 1. At 302, inputs to a call center agent device are recorded. As described above, these inputs can include a transcription of the call, a log of keystrokes made by the call center agent, tools or applications accessed by the agent using the agent device, and so on. At 304, the inputs are provided into an LLM. The inputs can be provided into the LLM for the LLM to identify the context of the inquiry from the member contacting the agent, as well as to identify what information is needed to respond to the inquiry.

At 306, the database(s) containing the information that may be relevant to responding to the inquiry are accessed (e.g., by the ANN). For example, the plan database can be accessed responsive to the inquiry seeking information on benefit eligibility. At 308, the information responsive to the inquiry can be obtained from the databases that are accessed. Following 308, one or more operations of 310, 312, 314, and/or 316 can be performed. At 310, a summary of the call between the member device and the agent device can be prepared and provided to the agent for validation. If the agent approves the summary as accurately reflecting the call, then at 312 a call log may be populated with this summary (and saved in one or more of the databases). This can assist with more efficiently handling a same or similar inquiry from the same or different member at a later time.

At 314, signals are generated and communicated to the call center agent device with the responsive information. The ANN can send the information responsive to the inquiry to the agent device, which can then present or provide the information to the member or member device. At 316, one or more forms, such as claim denial appeal forms, can be automatically completed and submitted. The ANN can obtain, complete, and submit these forms to the benefit manager or benefit manager system for the medical procedure and/or medication (for which the benefit claim was previously denied). This can allow the member to receive the medical procedure and/or medication with the benefit(s).

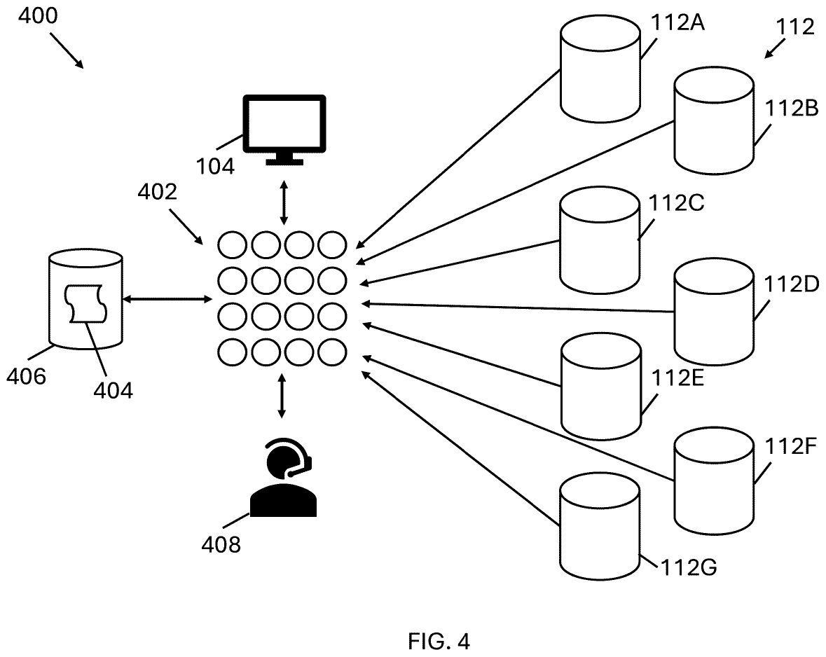

FIG. 4 illustrates one example of a healthcare personal concierge system 400. The concierge system 400 can be used to track many or all healthcare interactions for a member of a benefit plan, and to use information recorded during those interactions in a proactive manner to assist the member in future healthcare interactions or needs.

The concierge system 400 includes an ANN 402 that can interact with the member device 104, a manager device 408, and the databases 112. The ANN 402 also can interact with an LLM 404 stored in another database 406 as described herein. The ANN 402 can monitor data transactions and interactions between the member (e.g., the member device 104) and a benefit plan (e.g., the benefit manager device 408, which can represent hardware circuitry that includes and/or is connected with one or more processors for performing the operations described in connection with the device 408) over time. For example, during enrollment in the benefit plan, the ANN 402 can record (or obtain) demographic information about the member and/or the family of the member, eligibility information, and the like, and record the same information in the LLM 404 in the database 406. This information can be recorded directly from the member device 104, the manager device 408, or may be obtained from one or more of the databases 112 described herein.

As time continues, the member will have continued interactions with the benefit manager directly or indirectly, such as by submitting claims for coverage of medical procedures, provider visits, medication prescriptions, and the like. The ANN 402 can use the deep history of data maintained over time from usage of the benefit plan and interactions between the member and the benefit manager, providers, pharmacies, etc. to assist the member with future interactions.

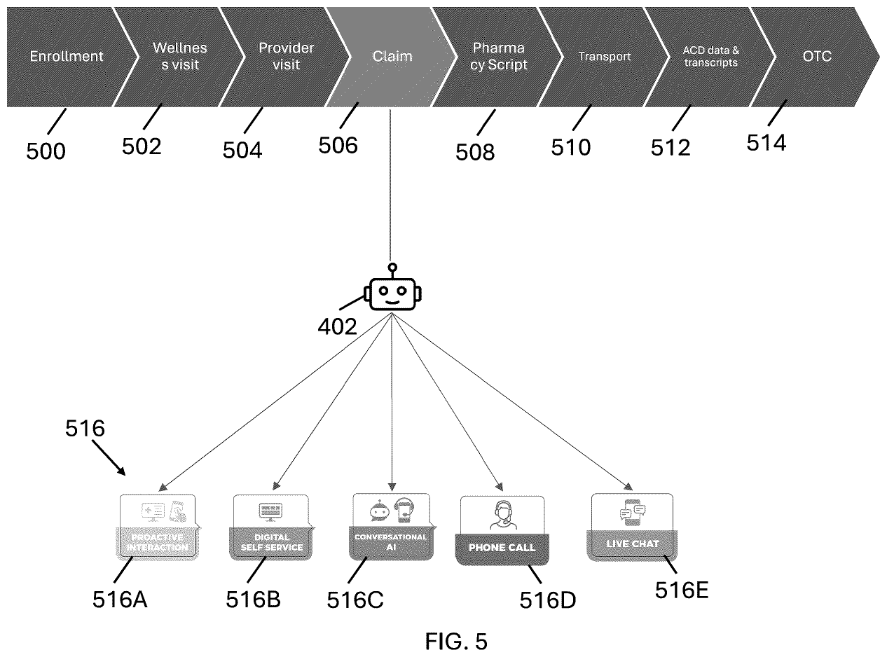

FIG. 5 illustrates one example of operation of the concierge system 400 over time. The ANN 402 can collect information sent to and/or from the member device 104 during enrollment 500 of the member within a benefit plan, during wellness visits 502 (e.g., dates, times, and locations of the visits; provider notes from the visits; medical test results from the visits; etc.), during provider visits 504 (e.g., dates, times, and locations of the visits; provider notes from the visits; medical test results from the visits; etc.), when claims benefits for medical procedures 506 are submitted (e.g., for medical procedures), when claims for prescriptions 508 are submitted (e.g., pharmacy scripts), when claims for transportation to/from medical visits or procedures 510 are submitted (e.g., transport), when calls for assistance 512 are made by the member (e.g., to a cell center operations system 100), when over the counter (OTC) 514 medications or the like are purchased, etc.

The data that can be collected can include the demographic information of the member, the claims data, details about the medications or procedures, information about the benefits that were provided or denied, the reasons that a benefit claim was approved or denied, preauthorization requirements, the information included in the preauthorization request, reasons why a preauthorization request was denied, call recordings, other recorded inputs to the call operations system 100, and the like.

The ANN 402 generate or update a ledger stored in the database 406 that indicates the data that is recorded. This data can be referred to as pre-aggregated healthcare data. This ledger can be repeatedly updated over time as the member engages in one or more additional healthcare provider visits, submits additional benefit claims, obtains additional OTC medications, or the like. The ANN 402 can then operate as a personal concierge to the member to assist the member in various aspects using the data included in the ledger. For example, a claim 506 for a benefit under the benefit plan may need to be submitted. The ANN 402 can identify this claim 506 by monitoring the ledger for new appointments made by the member, for claims data obtained from the claims database, etc.

The ANN 402 can then examine the ledger and/or other data in the databases 112 to identify a support channel 516 from among several different support channels 516A-E. The support channels 516 can represent different technological paths that can be taken by the member to obtain assistance (e.g., from the benefit manager) in receiving benefits associated with the claim 506. The support channel 516A can represent a proactive interaction channel 516A that involves the ANN 402 automatically submitting the pre-aggregated healthcare data to the pharmacy benefit plan manager to adjudicate the claim, obtain preauthorization for a procedure or medication, or appeal a denial of the claim. For example, the ANN 402 can select the information from the pre-aggregated healthcare data that is required to adjudicate the claim (as indicated by the eligibility data stored in the database 112F) and automatically submit this information to the benefit manager on behalf of the member.

The support channel 516B can represent a digital self-service channel that provides one or more forms or queries to the member for adjudicating the claim or appealing a denial of the claim. This channel 526B can include the ANN 402 providing one or more forms, websites, tools, or other applications to the member device 104 (or links to the same to the member device 104). The member can then submit the information required by the forms, websites, etc., to adjudicate the claim.

The support channel 516C can represent a conversational AI channel that engages in a text-based conversation with the member to adjudicate the claim or appeal a denial of the claim. For example, the ANN 406 or another ANN can use an LLM model (e.g., the LLM model 404) to engage in a conversation with the member via the member device 104. This conversation may allow the ANN 406 or other ANN to query the member via the member device 104 for information needed to adjudicate, appeal, or obtain preauthorization for the claim. The information that is then obtained can be uploaded to the benefit manager, such as via the member device 408 of the benefit manager, to handle the claim.

The support channel 516D can represent a phone call channel that automatically connects the member device 104 (or another device) of the member with a network that connects with the agent device 102 of the benefit manager to adjudicate the claim or appeal a denial of the claim. For example, instead of connecting the member with the conversational AI channel 516C, the ANN 402 may automatically establish a call between the agent device 102 or the member device 408, and the member device 104 to resolve the claim.

The support channel 516E can represent a live chat channel that automatically connects the member device 104 with a network that connects with the member device 408 or the agent device 102 of the benefit manager to engage in a text chat to handle the claim (e.g., adjudicate the claim or appeal a denial of the claim).

Different factors may be considered by the ANN 402 in deciding which of the support channels 516 to recommend to the member. For example, the member may have previously recorded or expressed preferences for one support channel 516 over others. Some members may prefer speaking with the call center agent via the channel 516D while others may prefer texting or chatting online without a phone call (e.g., via the channel 516C and/or 516E). Another factor may be the context of the issue related to adjudication of the claim. For example, some claims may be more complex to resolve to obtain the benefit, such as claims requiring preauthorization or preauthorization information from more than one healthcare provider. These claims may be handled via the channel 516D or 516E to ensure that the member is able to speak with a person at the call center to handle the claims. Another factor is the success rates of the different support channels 516. For example, some claims (e.g., pharmacy claims) may have greater rates of success in adjudicating the claims in favor of granting benefits to the member when handled by the channel 516A instead of other channels 516. Conversely, some more complex claims (e.g., those requiring pre-authorization or claims requiring an appeal) may have greater success rates using more interpersonal contact (e.g., the channels 516D, 516E) instead of more individual-use channels (e.g., the channel 516B).

The ANN 402 may assist the member in resolving the claim using the pre-aggregated healthcare data. As described above, the ANN 402 can automatically obtain the data needed to adjudicate a claim, file an appeal of a claim denial, obtain preauthorization for a medication or procedure, or the like. The ANN 402 can operate as a personal concierge to the member by either pointing the member to the best support channel 516 for resolving any issues with a claim and/or for automatically assisting or handling the claim on behalf of the member.

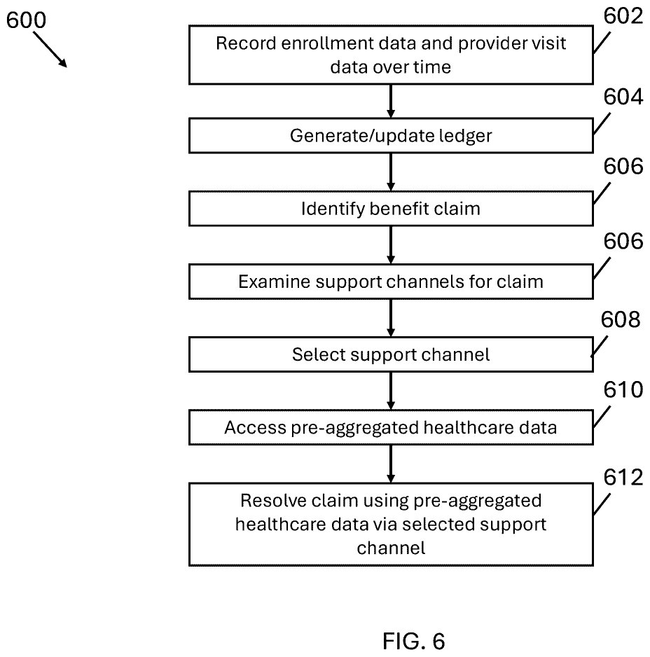

FIG. 6 illustrates a flowchart of one example of a method 600 for providing a personal healthcare data concierge. The method 600 can represent one or more operations performed by the ANN 402 shown in FIG. 4. At 602, healthcare-related data is recorded over time. This can begin with the data involved in enrolling a member in a benefit plan, and extend to claims data, data from provider visits, etc. At 604, a ledger personalized for the member is generated or updated. This ledger can represent the healthcare-related data associated with the member. The ledger can include the healthcare-related data, as well as the call logs stored in the database 112G described above.

At 606, a benefit claim is identified. This can occur when the member attempts to obtain benefits under a benefit plan for a medication, procedure, provider visit, or the like. At 608, several different support channels can be examined for helping the member adjudicate the claim in favor of the member and, at 610, a support channel is selected. As described above, different factors can be considered when selecting the support channel. At 610, the pre-aggregated healthcare data is accessed. This can be accessed in the ledger to assist the member in resolving the claim. At 612, the claim is resolved using the healthcare data via the support channel that is selected. As described above, the channel can assist the member to handle the claim or handle the claim automatically without additional input from the member to assist the member.

In one example, a computer-implemented method for call center operations of a pharmacy benefit manager is provided. The method can include recording inputs into a call center agent device from one or more of a member of a pharmacy benefit plan provided by the pharmacy benefit manager or a call center agent handling a call or computer-implemented chat from the member. The inputs can include one or more inquiries from the member regarding one or more benefits provided by the pharmacy benefit plan. The method also can include providing the inputs that are recorded into a LLM, accessing (using the LLM) several different healthcare-related databases storing data related to the member and the pharmacy benefit plan, identifying (using the LLM) one or more sets of responsive information from the healthcare-related databases based on the inputs, and communicating one or more electronic signals from the LLM to the call center agent device to convey the one or more sets of responsive information to the call center agent device. The one or more sets of responsive information are provided to the call center agent device to prevent the call center agent from having to search through and find the responsive information from the different healthcare-related databases.

Optionally, the method also can include automatically aggregating benefit claims data from the healthcare-related databases using the LLM as the one or more sets of responsive information. The method also can include automatically completing, using the LLM, one or more appeal forms for appealing a denial of a claim for benefit under the pharmacy benefit plan based on the one or more sets of responsive information that are obtained.

The method also can include automatically submitting the one or more appeal forms to the pharmacy benefit manager using the LLM to appeal the denial of the claim on behalf of the member. Recording the inputs, providing the inputs, accessing the healthcare-related databases, identifying the one or more sets of responsive information, and communicating the one or more electronic signals can occur during training of the call center agent.

The healthcare-related databases can include one or more of a benefit claims database storing medical claims data, a dental claims database storing dental benefit claims data, a provider database storing healthcare provider data, a member database storing member demographic data, a pharmacy database storing pharmacy claims data, or a plan database storing benefit plan eligibility data.

The method also can include recording communications between the call center agent and the member during the call, inputting the communications that are recorded into the LLM, generating a summary of the communications and the one or more sets of responsive information using the LLM, directing a display device to present the summary to the call center agent, receive a validation action from the call center agent responsive to directing the display device to present the summary, and automatically populating a call log with the summary responsive to receiving the validation action.

In another example, an ASIC for an ANN is provided. The ASIC includes an LLM that includes neurons organized in an array, each of the neurons including a register, a processing element, and at least one input; and synaptic circuits, each of the synaptic circuits including a memory for storing a synaptic weight. Each of the neurons is connected to at least one other of the neurons via at least one of the synaptic circuits. The processing elements of the neurons configured to record inputs into a call center agent device from one or more of a member of a pharmacy benefit plan provided by a pharmacy benefit manager or a call center agent handling a call or computer-implemented chat from the member, the inputs including one or more inquiries from the member regarding one or more benefits provided by the pharmacy benefit plan; access several different healthcare-related databases storing data related to the member and the pharmacy benefit plan; identify one or more sets of responsive information from the healthcare-related databases based on the inputs; and communicate one or more electronic signals from the LLM to the call center agent device to convey the one or more sets of responsive information to the call center agent device, wherein the one or more sets of responsive information are provided to the call center agent device to prevent the call center agent from having to search through and find the responsive information from the different healthcare-related databases.

The processing elements can automatically aggregate benefit claims data from the healthcare-related databases using the LLM as the one or more sets of responsive information. The processing elements can automatically complete one or more appeal forms for appealing a denial of a claim for benefit under the pharmacy benefit plan based on the one or more sets of responsive information that are obtained.

The processing elements can automatically submit the one or more appeal forms to the pharmacy benefit manager using the LLM to appeal the denial of the claim on behalf of the member. The processing elements can record the inputs, access the healthcare-related databases, identify the one or more sets of responsive information, and communicate the one or more electronic signals during training of the call center agent.

The healthcare-related databases can include one or more of a benefit claims database storing medical claims data, a dental claims database storing dental benefit claims data, a provider database storing healthcare provider data, a member database storing member demographic data, a pharmacy database storing pharmacy claims data, or a plan database storing benefit plan eligibility data.

The processing elements can record communications between the call center agent and the member during the call; generate a summary of the communications and the one or more sets of responsive information; direct a display device to present the summary to the call center agent; receive a validation action from the call center agent responsive to directing the display device to present the summary; and automatically populate a call log with the summary responsive to receiving the validation action.

In another example, an ASIC for an ANN that provides an automated healthcare data concierge is provided. The ASIC includes neurons organized in an array, each of the neurons including a register, a processing element, and at least one input; and synaptic circuits, each of the synaptic circuits including a memory for storing a synaptic weight. Each of the neurons is connected to at least one other of the neurons via at least one of the synaptic circuits. The processing elements of the neurons can record data from enrollment of a member into a pharmacy benefit plan and one or more healthcare provider visits by the member over time; generate or update a ledger indicative of the data that is recorded, wherein the ledger is updated over time as the member engages in one or more additional healthcare provider visits; identify a claim for a benefit pursuant to the pharmacy benefit plan for the member; identify a support channel among plural different support channels for resolving the claim based on one or more of a recorded preference of the member, a context of an issue related to adjudication of the claim, or success rates of the different support channels; access pre-aggregated healthcare data useful for resolving the claim based on the support channel that is identified; and automatically resolving the claim using the pre-aggregated healthcare data.

The processing elements can identify the support channel as a proactive interaction channel that involves the processing elements automatically submitting the pre-aggregated healthcare data to the pharmacy benefit plan to adjudicate the claim or appeal a denial of the claim. The processing elements can identify the support channel as a digital self-service channel that provides one or more forms or queries to the member for adjudicating the claim or appealing a denial of the claim.

The processing elements can identify the support channel as a conversational AI channel that engages in a text-based conversation with the member to adjudicate the claim or appeal a denial of the claim. The processing elements can identify the support channel as a phone call channel that automatically connects a telephone device of the member with a network that connects the telephone device with an agent of a pharmacy benefit manager to adjudicate the claim or appeal a denial of the claim. The processing elements can identify the support channel as a live chat channel that automatically connects a computer device of the member with a network that connects the computer device with an agent device of a pharmacy benefit manager to engage in a text chat to adjudicate the claim or appeal a denial of the claim.

The present embodiments can provide methodology for overarching persistent data observational processes with the LLM monitoring data updates to assure compliance and adherence to specific plan limits (e.g., fixed variables in a database) and service levels (e.g., fixed variables in a database). These improve the performance of a computing system based on the requirements of individual agreements.

The present systems and methods (e.g., an ANN) can use the trends of any data type described herein. A trend can include the value over a time period. The present LLM can use advanced deep learning, e.g., short-term memory networks, to recognize patterns in sequential data (e.g., the trend). Hence, the model may be trained using hundreds or thousands of first level variables, and then create second level variables (e.g., trends), and may also create third or further level variables. This number of variables is too large for a person to calculate by hand to discover new connections and weighting of variables within the model for meaningful results.

Further examples of call center systems that can be used with the present embodiments are described in U.S. Pat. Nos. 11,445,068, filed 21 Feb. 2020; U.S. Pat. No. 11,315,065, filed 19 Dec. 2019; U.S. Pat. No. 11,039,014, filed 13 Jun. 2019; and U.S. Pat. No. 11,087,880, filed 20 Jul. 2017, which are all hereby incorporated by reference.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

It is to be understood that the above description is intended to be illustrative, and not restrictive. For example, the above-described examples (and/or aspects thereof) can be used in combination with each other. In addition, many modifications can be made to adapt a particular situation or material to the teachings of the various examples of the disclosure without departing from their scope. While the dimensions and types of materials described herein are intended to define the aspects of the various examples of the disclosure, the examples are by no means limiting and are exemplary examples. Many other examples will be apparent to those of skill in the art upon reviewing the above description. The scope of the various examples of the disclosure should, therefore, be determined with reference to the appended claims, along with the full scope of equivalents to which such claims are entitled. In the appended claims and the detailed description herein, the terms “including” and “in which” are used as the plain-English equivalents of the respective terms “comprising” and “wherein.” Moreover, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various examples of the disclosure, including the best mode, and also to enable any person skilled in the art to practice the various examples of the disclosure, including making and using any devices or systems and performing any incorporated methods. The patentable scope of the various examples of the disclosure is defined by the claims, and can include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

1.-20. (canceled)

21. A call center process, comprising:
recording audio inputs at the call center agent device from members, call center agents, or both;
providing the audio inputs to a large language model (LLM) circuitry in an artificial neural network;
processing the inputs by the LLM circuitry to develop LLM data requests;
accessing a plurality of healthcare-related databases using the LLM circuitry and containing information for the LLM data requests;
identifying information from the plurality of databases responsive to the LLM data requests; and
communicating the information back to the call center agent device to prevent the agent from manually searching through different databases in the plurality of databases.

22. The call center process of claim 21, wherein communicating the information back to the call center device includes communicating a predefined set of actions from the LLM circuitry based on a context of the audio inputs based on the LLM data requests and the information communicated back to the call center agent device.

23. The call center process of claim 22, wherein processing the inputs by the LLM circuitry includes inputting the audio inputs to a first level of neurons in the LLM circuitry, storing the audio inputs in a register of the neuron, processing the audio input to produce an output for each neuron, sending the output over a synaptic circuit, applying a synaptic weight to the output at the synaptic circuit, and receiving the weighted output at a register of a subsequent neuron.

24. The call center process of claim 21, wherein identifying information from the plurality of databases responsive to the LLM data requests includes auto-completing a preauthorization form using the identified information and communicating includes sending the completed preauthorization form to the call center agent device.

25. The call center process of claim 21, wherein identifying information from the plurality of databases responsive to the LLM data requests includes auto-completing a healthcare appeal form using the identified information and communicating includes sending the completed healthcare appeal form to the call center agent device.

26. The call center process of claim 21, wherein identifying information from the plurality of databases responsive to the LLM data requests includes populating a call log in a call log database with the information identified by the LLM circuitry.

27. The call center process of claim 21, wherein providing the audio inputs to the LLM circuitry includes providing additional call center agent device data from the call center agent device with the audio inputs to the LLM circuitry and providing the additional call center agent device data to the LLM circuitry to determine which of the plurality of databases include information responsive to a call center interaction between the member and the call center agent.

28. The call center process of claim 27, wherein the additional call center agent device data includes at least one of text, keystrokes, electronic mouse inputs, electronic stylus inputs, detected touches on a touchscreen, or combinations thereof.

29. The call center process of claim 21, wherein accessing the plurality of healthcare-related databases using the LLM circuitry includes accessing a member database, a prescription database, a benefit claim database, and a provider database.

30. The call center process of claim 21, wherein communicating the information back to the call center agent device includes displaying a single interface on the call center agent device responsive to a caller issue of the recorded audio inputs.
